(19) United States
(12) Patent Application Publication
(10) Pub. No.: US 2007/0053968 A1
(43) Pub. Date: Mar. 8, 2007

(54) Transdermal drug delivery devices containing O-Desmethyl Venlafaxine (ODV) or its salts

(75) Inventors: Rao Tatapudy, Suffern, NY (US); Syed M. Shah, East Hanover, NJ (US)

Correspondence Address: CHOATE, HALL & STEWART LLP, PATENT GROUP, TWO INTERNATIONAL PLACE, BOSTON, MA, 02110, US

(73) Assignee: Wyeth, Madison, NJ (US)

(21) Appl. No.: 11/515,708

(22) Filed: Sep. 5, 2006

Related U.S. Application Data
(60) Provisional application No. 60/714,582, filed on Sep. 7, 2005.

(51) Int. Cl. A61K 9/70; A61K 31/138

(52) U.S. Cl. 424/449; 514/651

(57) ABSTRACT

The present invention provides transdermal drug delivery devices (i.e., patches) comprising O-desmethylvenlafaxine (ODV), a selective serotonin and norepinephrine re-uptake inhibitor, or a pharmaceutically acceptable salt thereof, which, among other, offer the advantage of eliminating or reducing the adverse side effects associated with the oral administration of ODV. Also provided are methods of preparing and using these transdermal delivery systems for the treatment of depression, anxiety disorders, vasomotor symptoms and pain.

### RELATED APPLICATIONS

The present application claims priority from Provisional Application No. 60/714,582 filed on Sep. 7, 2005 and entitled “Transdermal Drug Delivery Devices Containing O-Desmethyl Venlafaxine (ODV) or Its Salts”. The Provisional Application is incorporated herein by reference in its entirety.

### BACKGROUND OF THE INVENTION

Venlafaxine (or (±)-1-[2-(dimethylamino)-1-(4-methoxyphenyl)ethyl]-cyclohexanol) belongs to a relatively new class of anti-depressants (U.S. Pat. No. 4,761,501; J. T. Pento, Drugs of the future, 1988, 13: 839-840). Its hydrochloride salt is commercially available in the U.S. under the trade name Effexor® and is currently indicated for the treatment of depression and anxiety disorders.

In vivo, venlafaxine is extensively transformed by a saturable metabolic pathway into two minor metabolites, N-desmethylvenlafaxine and N,O-didesmethylvenlafaxine, and one major, biologically active metabolite, O-desmethylvenlafaxine (K. J. Klamerus et al., J. Clin. Pharmacol., 1992, 32: 716-724). Venlafaxine and O-desmethylvenlafaxine (ODV) are structurally unrelated to other anti-depressant drugs including tricyclic anti-depressants (TCAs), selective serotonin re-uptake inhibitors (SSRIs), monoamine oxidase inhibitors (MAOIs) and reversible inhibitors of monoamine oxidase (RIMAs). The mechanism of anti-depressant action of venlafaxine and ODV in humans is associated with their potentiation of neurotransmittor activity in the central nervous system; they have been shown to be potent inhibitors of neuronal serotonin and norepinephrine re-uptake and weak inhibitors of dopamine re-uptake. Selective serotonin and norepinephrine re-uptake inhibitors, or “SSNRIs”, i.e., compounds that exert their anti-depressant effect through the same mechanism as venlafaxine, have, in general, a quicker onset of therapeutic action and are usually more effective than other anti-depressants (J. S. Olver et al., CNS Drugs, 2001, 15: 941-954; M. E. Thase, J. Clin. Psychiatry, 64: 3-7; D. E. Stewart, J. Clin. Psychiatry, 2003, 64: 12-16). Furthermore, since Venlafaxine and ODV exhibit no significant affinity for muscarinic, H1-histaminergic or α1-adrenergic receptors, they are not associated with the various anticholinergic, sedative, and cardiovascular effects seen with other anti-depressant drugs.

Compared to venlafaxine, ODV possesses several advantageous properties. In addition to being more soluble than venlafaxine, ODV has been reported to have a half-life of about 10 hours, which is approximately 2.5 times as long as that of the parent compound (K. J. Klamerus et al., J. Clin. Pharmacol., 1992, 32: 716-724). In vitro studies suggest that ODV is also a more potent inhibitor of norepinephrine and serotonin re-uptake than venlafaxine (E. A. Muth et al., Drug Develop. Res., 1991, 23: 191-199). These advantages are all the more important given that ODV, like venlafaxine, can find applications in the treatment of other conditions than major depression.

For example, venlafaxine is known to be effective in treating obsessive-compulsive conditions, post-traumatic stress disorder, panic disorder, and other anxiety disorders (T. T. Pleak and L. J. Gormly, Am. J. Psychiatry, 1995, 152: 1099; T. D. Geracioti, J. Clin. Psychiatry, 1995, 56: 408-410; J. A. Yaryura-Tobias and F. A. Neziroglu, Arch. Gen. Psychiatry, 1996, 53: 653-654; D. Denys et al., J. Clin. Psychopharmacol., 2003, 23: 568-575; R. H. Bradley et al., Am. J. Ther., 2003, 10: 318-323; M. Katzman, Expert Rev. Neurother., 2004, 4: 371-381). Anti-depressants, such as venlafaxine, that block re-uptake of both serotonin and norepinephrine have also been used to treat pain syndromes including, but not limited to, pain associated with major depression or an anxiety disorder (R. H. Bradley et al., Am. J. Ther., 2003, 10: 318-323); peripheral neuropathic pain (J. E. Sumpton and D. E. Moulin, Ann. Pharmacother., 2001, 35: 557-559; T. Tasmuth et al., Eur. J. Pain, 2002, 6: 17-24; S. Guldiken et al., Diabetes Nutr. Metab., 2004, 17: 247-249); chronic pain (K. Taylor and M. Rowbowtham, West. J. Med., 1996, 165: 147-148; D. A. Songer and H. Schulte, Am. J. Psychiatry, 1996, 153: 737; P. T. Ninan, Depress. Anxiety, 2000, 12: 90-94); cancer-related pain (J. P. Durand and F. Goldwasser, Anticancer Drugs, 2002, 13: 777-780; J. P. Durand et al., Anticancer Drugs, 2003, 14: 423-425; S. S. Reuben et al., J. Pain Symptom Manag., 2004, 27: 133-139), and fibromyalgia (M. M. Dwight et al., Psychosomatics, 1998, 39: 14-17; K. Sayar et al., Ann. Pharmacother., 2003, 37: 1561-1565). Venlafaxine is also considered as a promising non-hormonal alternative for relief of vasomotor symptoms (VMS) including hot flashes (C. L. Loprinzi et al., J. Clin. Oncol., 1998, 16: 2377-2381; S. K. Quella et al., J. Urol., 1999, 162L 98-102; D. H. Barlow, Lancet, 2000, 356: 2025-2026; C. L. Loprinzi et al., Lancet, 2000, 356: 2059-2063; D. Barton et al., Oncol. Nurs. Forum, 2002, 29: 33-40; A. N. Wymenga and D. T. Sleijfer, Acta Oncol., 2002, 41: 269-275; C. E. Schober and N. T. Ansani, Ann. Pharmacother., 2003, 37: 1703-1707), and ODV succinate is currently in Phase III clinical trials for VMS.

However, oral administration of venlafaxine is associated with adverse side effects including sustained hypertension, headache, asthenia, sweating, somnolence, dry mouth, dizziness, insomnia, nervousness, anxiety, blurred or blurry vision, sexual dysfunction (Physician's Desk Reference, 1999, 53^{rd} Ed, pp. 3293-3302; J. Sinclair et al., Rev. Contemp. Pharmacother., 1998, 9: 333-344), and, most commonly, gastrointestinal side effects such as nausea and vomiting (R. Entsuah and R. Chitra, Psychopharmacol. Bull., 1997, 33: 671-676). These adverse effects can significantly limit the dose level, frequency, and duration of treatment, and can even prevent the potential of such drugs from being fully realized.

There clearly exists a need for novel strategies for the administration of selective serotonin and norepinephrine re-uptake inhibitors, such as ODV. Particularly desirable are delivery systems that would allow administration of therapeutically effective amounts of SSNRIs while avoiding or reducing the incidence, severity or duration of the undesired side effects generally associated with their oral administration.

### SUMMARY OF THE INVENTION

The present invention is directed to systems and methods for the simple, convenient and non-invasive administration of ODV or its salts. More specifically, the present invention provides transdermal drug delivery devices (i.e., patches) containing ODV compositions, which offer the advantage of avoiding the gastrointestinal tract and hepatic first-pass biotransformation and metabolism. In particular, the inventive transdermal patches allow for the rapid delivery of high concentrations of the drug to affected tissues, which results in fewer adverse side effects or drug-drug interactions than oral administration. The transdermal ODV patches of the present invention can be used for the treatment of a wide variety of diseases or conditions including, but not limited to, major depressive disorder, anxiety disorders, vasomotor symptoms and pain.

More specifically, in one aspect, the present invention provides a transdermal patch for the administration of a topical composition, the topical composition comprising a therapeutically effective amount of ODV, or a pharmaceutically acceptable salt thereof, and at least one physiologically acceptable carrier or excipient. The physiologically acceptable carrier or excipient may be selected from the group consisting of tromethane ethanol, polyethylene glycol, glycerin, propylene glycol, acrylates, Carbopol, purified water, benzyl alcohol, cetyl alcohol, citric acid, monoglycerides, diglycerides, triglycerides, oleyl alcohol, sodium cetostearylsulphate, sodium hydroxide, stearyl alcohol, white petrolatum, mineral oil, propylene carbonate, white wax, paraffin, and any combination thereof. The topical composition may further comprise at least one absorption enhancer, such as pentadecalactone, 1,3-dioxalanes, 1,3-dioxanes, or any combination thereof.

In certain embodiments, the topical composition of the inventive transdermal patch further comprises a therapeutically effective amount of at least one additional pharmacologically active agent. The pharmacologically active agent may be selected from the group consisting of analgesics, anesthetics, muscle relaxants, neurotransmitter regulating agents, nociceptic agents, pre-menstrual medications, anti-menopausal agents, anti-aging agents, anti-anxiolytic agents, mood disorder agents, anti-depressants, anti-bipolar agents, anti-schizophrenic agents, tranquilizers, soporific agents, anti-migraine agents, skin temperature lowering products, anti-cancer agents, alkaloids, anti-metastatic agents, blood pressure controlling agents, hormones, steroids, anti-inflammatory agents, anti-ischemic agents, anti-arrhythmic agents, vitamins, minerals, anti-angiogenic agents, wound healing agents, cytokines, growth factors, anti-histaminic agents, anti-bacterial agents, anti-viral agents, antibiotics, counteracting appetite suppressants, dermatological agents such as skin renewal agents, sun screen and emollients, libido altering agents, laxatives, anti-diarrheic agents, antipruritic agents, antipyretic agents, immunostimulating agents, agents suitable for the treatment of prophylaxis diseases and conditions associated or accompanied with pain and inflammation, and any combination thereof.

In certain embodiments, the transdermal patch is a reservoir patch, a matrix patch or a drug-in-adhesive patch, and optionally comprises a release liner.

In some embodiments, the therapeutically effective amount of ODV, or a pharmaceutically acceptable salt thereof, contained in the transdermal patch is between about 5 mg and about 500 mg, between about 25 mg and about 250 mg, or between about 50 mg and about 200 mg, wherein the amount is calculated based on the amount of ODV free base. In certain preferred embodiments, the therapeutically effective amount of ODV, or a pharmaceutically acceptable salt thereof, is about 100 mg.

In another aspect, the present invention provides a method for treating a depression disorder in a subject, the method comprising applying a transdermal patch as described above to the skin surface of the subject for a period of time effective to treat the depression disorder. The depression disorder may be major depressive disorder, depression in cancer patients, depression in Parkinson's patients, postmyocardial infarction depression, subsyndromal symptomatic depression, depression in infertile women, single episode depression, recurrent depression, child abuse induced depression and, or post-partum depression. In certain embodiments, the period of time effective to treat the depression disorder may be about 1 week to about 1 month.

In another aspect, the present invention provides a method for treating an anxiety disorder in a subject, the method comprising applying a transdermal patch as described above to the skin surface of the subject for a period of time effective to treat the anxiety disorder. The anxiety disorder may be generalized anxiety disorder, phobias, agoraphobia, social phobia, simple phobias, post-traumatic stress, syndrome, acute stress disorder, avoidant personality disorder, eating disorders, anorexia nervosa, bulimia nervosa, obesity, obsessive compulsive disorder, panic disorder, premenstrual syndrome, or attention deficit disorder. In certain embodiments, the period of time effective to treat the anxiety disorder may be about 1 week to about 1 month.

In still another aspect, the present invention provides a method for treating vasomotor symptoms in a subject, the method comprising applying a transdermal patch as described above to the skin surface of the subject for a period of time effective to treat the vasomotor symptoms. The subject suffering from vasomotor symptoms may experience hot flashes. In certain embodiments, the period of time effective to treat vasomotor symptoms may be about 30 minutes to about 3 hours.

For example, this method of the invention may be used to treat a female patient experiencing vasomotor symptoms associated with natural menopause, chemically-induced menopause or surgically-induced menopause. Alternatively or additionally, the inventive method may be used to treat a female patient who is receiving or has received breast cancer treatment, such as for example a treatment comprising administration of tamoxifen. The inventive method may also be used to treat a male patient who is naturally, chemically or surgically andropausal. Alternatively or additionally, the method may be used to treat a male patient who is being or has been treated for prostate cancer.

In yet another aspect, the present invention provides a method for treating pain in a subject, the method comprising applying a transdermal patch as described above to the skin surface of the subject for a period of time effective to treat the pain. The transdermal patch may be applied to the skin surface adjacent to the subject's body site experiencing pain. In certain embodiments, the period of time effective to treat the pain may be about 1 hour to about 1 month. The pain may be nociceptive pain or neuropathic pain.

These and other objects, advantages and features of the present invention will become apparent to those of ordinary skill in the art having read the following detailed description of the preferred embodiments.

### DEFINITIONS

Throughout the specification, several terms are employed that are defined in the following paragraphs.

The terms “individual”, “subject” and “patient” are used herein interchangeably. They refer to a higher vertebrate, preferably a human or another mammal (e.g., mice, rats, other rodents, rabbits, dogs, cats, cattle, swine, sheep, horses, or primates).

The terms “patch”, “skin patch”, and “adhesive skin patch” are used herein interchangeably. They refer to a drug delivery device comprising, at least, a topical formulation and a covering layer, such that, the patch can be placed over the area of skin to be treated. Preferably, a patch is designed to maximize drug delivery through the stratum corneum and into the epidermis or dermis, to reduce lag time, promote uniform absorption, and reduce mechanical rub-off.

The terms “topical formulation” and “topical composition” are used herein interchangeably. They refer to a composition formulated such that the active ingredient(s) of the composition may be placed for application to a skin surface and from which an effective amount of the active ingredient(s) is released. Examples of topical formulations include, but are not limited to, ointments, creams, gels, lotions, sprays, pastes, and the like. In certain embodiments of the present invention, a patch comprises a topical composition of ODV, or a pharmaceutically acceptable salt thereof. An ODV topical composition preferably comprises at least one physiologically acceptable carrier or excipient and an effective amount of ODV or a pharmaceutically acceptable salt thereof.

The terms “skin” and “skin surface” are used herein interchangeably. They encompass the skin surface of a subject comprising the epidermis as well as mucosal surfaces to which a transdermal drug delivery device of the present invention may be applied. Examples of mucosal surfaces include the mucosa of the respiratory, oral, vaginal, introital, labial, and rectal surfaces.

The term “transdermal” refers to the route of administration that facilitates transfer of the active ingredient(s) of a composition through a skin or mucosal surface and, optionally, into the bloodstream.

The terms “penetration enhancer”, “permeation enhancer” and “absorption enhancer” are used herein interchangeably. They refer to compounds or substances that increase the permeability of skin or mucosa to a pharmacologically active agent so as to increase the rate at which the agent permeates through the skin or mucosa and enters the bloodstream. Absorption enhancers and their use in topical formulations are well known in the art.

The term “ODV” refers to O-desmethylvenlafaxine (or 1-[2-(dimethyl-amino)-1-4-phenyl)ethyl]-cyclohexanol), the major metabolite of venlafaxine.

As used herein, the term “pharmaceutically acceptable salt of ODV” refers to any salt of ODV derived from organic or inorganic acids, such as, for example, acetic, lactic, citric, cinnamic, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic acid, and the like, that is not toxic to the host at the concentrations at which it is administered. Preferably, a pharmaceutically acceptable salt of ODV has similar or superior biological activity than ODV and/or venlafaxine. Alternatively or additionally, a pharmaceutically acceptable salt of ODV exhibits desirable properties for topical administration (e.g., improved percutaneous/permucosal penetration). The term “pharmaceutically acceptable salt of ODV” also encompasses pharmaceutically acceptable salt hydrates of ODV (i.e., salts of ODV associated with molecules of water).

As used herein, the term “physiologically acceptable carrier or excipient” refers to a carrier medium or an excipient which does not interfere with the effectiveness of the biological activity of the active ingredient(s) of the composition and which is not excessively toxic to the host at the concentrations at which it is administered. In the context of the present invention, a physiologically acceptable carrier or excipient is preferably suitable for topical formulation. The term includes, but is not limited to, solvents, dispersion media, isotonic agents, percutaneous/permucosal absorption enhancers, and the like. The use of such media and agents for the formulation of pharmaceutically active substances is well known in the art (see, for example, “Remington's Pharmaceutical Sciences”, E. W. Martin, 18^{th} Ed., 1990, Mack Publishing Co.: Easton, Pa., which is incorporated herein by reference in its entirety).

The term “treatment” is used herein to characterize a method that is aimed at (1) delaying or preventing the onset of a medical condition, disease or disorder; (2) slowing down or stopping the progression, aggravation, or deterioration of the symptoms of the condition; (3) bringing about ameliorations of the symptoms of the condition; and/or (4) curing the condition. The treatment may be administered prior to the onset of the condition, for a prophylactic or preventive action, or it may be administered after initiation of the condition, for a therapeutic action.

As used herein, the term “therapeutically effective amount” refers to an amount sufficient to achieve (in principle, for a subject of comparable characteristics, such as species, body type, size, extent of disease or disorder, degree or type of symptoms, history of responsiveness, and/or overall health) an intended biological or medical response or therapeutic benefit in a tissue, system or subject. For example, a desirable response may include one or more of: delaying or preventing the onset of a medical condition, disease or disorder, slowing down or stopping the progression, aggravation, or deterioration of the symptoms of the condition, bringing about ameliorations of the symptoms of the condition, and curing the condition. As will be appreciated by one skilled in the art, a therapeutically effective amount of ODV, or a pharmaceutically salt thereof, may be different depending on the desired response. For instance, an amount of ODV effective to treat pain may be different from an amount of ODV effective to treat vasomotor symptoms or depression. Similarly, an amount of ODV effective to prevent vasomotor symptoms may be different from an amount of ODV effective to treat vasomotor symptoms, and either may be different from amounts to prevent or treat pain. It will also be appreciated that an amount of ODV effective to treat a local condition (e.g., pain) may be different from an amount of ODV effective to treat a condition where systemic drug distribution is desired (e.g., depression).

Furthermore, when combinations of ODV and other therapeutic agents are administered through a patch of the present invention, the amount of any individual agent required in the combination may be different from the amount required of that agent to achieve its therapeutic effect alone. In some cases, synergies between or among therapeutic agents used in a combination may reduce amounts required; in other cases, inhibitory interactions may increase amounts required. Thus, in general, therapeutically effective amounts of a combination of agents may utilize different absolute amounts of the agents than what constitute therapeutically effective amounts of the agents individually.

As used herein, the term “co-administration” refers to administration of multiple biologically active substances to one subject, either simultaneously or sequentially. The term also refers to simultaneous or sequential administration of a single biologically active substance to one subject using different administration routes (e.g., orally and topically).

The term “about” is used herein to mean within 10%, preferably within 5%, and more preferably within 1% of a given value or range. Alternatively, the term “about” means within an acceptable standard error of the mean, when considered by one of ordinary skill in the art.

The term “hot flash” has herein its art understood meaning and refers to an episodic disturbance in body temperature typically consisting of a sudden skin flushing, usually accompanied with perspiration.

The terms “vasomotor symptoms”, “vasomotor instability symptoms” and “vasomotor disturbances” are used herein interchangeably and include, but are not limited to, hot flashes, insomnia, sleep disturbances, mood disorders, irritability, excessive perspiration, night sweats, fatigue, and the like, caused by thermoregulatory dysfunction.

As used herein, the term “pain” refers to any type of nociceptive pain or neuropathic pain, whether centralized or localized.

The term “depression”, as used herein, refers to a variety of clinical conditions characterized by low self-esteem, guilt, self-reproach, introversion, sadness, despair, sleeping disorders, eating disorders, and/or discouragement. The term includes, but is not limited to, depression disorders, for example, single episodic or recurrent major depressive disorders, and dysthymic disorders, depressive neurosis, and neurotic depression, melancholic depression including anorexia, weight loss, and insomnia, and psychomotor retardation, atypical depression (or reactive depression) including increased appetite, hypersomnia, psychomotor agitation or irritability, anxiety and phobias, seasonal affective disorder, or bipolar disorders or manic depression, for example bipolar I disorder, bipolar II disorder and cyclothymic disorder. Other mood disorders encompassed within the term “depression” include dysthymic disorder with early or late onset with or without atypical features; dementia of the Alzheimer's type with depressed mood; vascular dementia with depressed mood, mood disorders induced by alcohol, amphetamines, cocaine, hallucinogens, inhalants, opioids, sedatives, hypnotics, anxiolytics and other substances; schizoaffective disorder of the depressed type; and adjustment disorders with depressed mood. The term also includes depression in cancer patients, depression in Parkinson's patients, postmyocardial infarction depression, depression associated with menopause, depression in infertile women, pediatric depression, child abuse induced depression and post-partum depression.

As used herein, the term “anxiety” includes anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalized anxiety disorder. The term “generalized anxiety” is typically defined as an extended period (e.g., at least six months) of excessive anxiety or worry with symptoms on most days of that period. The anxiety and worry is difficult to control and may be accompanied by restlessness, being easily fatigued, difficulty concentrating, irritability, muscle tension, and disturbed sleep.

### DETAILED DESCRIPTION OF CERTAIN PREFERRED EMBODIMENTS

As mentioned above, the present invention provides transdermal drug delivery devices comprising ODV, or a pharmaceutically acceptable salt thereof, which are useful in the prevention, treatment or management of a variety of diseases and conditions including depression, anxiety disorders, vasomotor symptoms and pain.

I—ODV and Pharmaceutically Acceptable Salts Thereof

In certain embodiments, the transdermal drug delivery devices of the present invention comprise ODV as active ingredient. In other embodiments, the active ingredient is a pharmaceutically acceptable salt of ODV.

ODV free base is a colorless solid; its preparation and physicochemical characteristics have been described in International Patent Applications WO 00/32555 and WO 00/59851 (each of which is incorporated herein by reference in its entirety).

ODV contains an asymmetric carbon atom. Accordingly, in the transdermal patches of the present invention, ODV may be present as the racemic mixture, as a non-equimolar mixture of the (+) and (−) enantiomeric forms of ODV, as the stereoisomerically pure (+) enantiomer or as the stereoisomerically pure (−) enantiomer. The term “stereoisomerically pure”, as used herein, refers to compounds which are comprised of a greater proportion of the desired isomer than the racemic mixture. A stereoisomerically pure compound is preferably made up of at least about 90% of the desired isomer, more preferably of at least 95% of the desired isomer, even more preferably of more than 97% of the desired isomer.

Preferred salts for use in the preparation of transdermal patches according to the present invention are pharmaceutically acceptable acid addition salts of ODV. These salts may be prepared by conventional methods which are well known in the art, for example, by reacting ODV free base with an equivalent amount of any acid that leads to the formation of a non-toxic salt. Suitable acids include organic and inorganic acids, such as, for example, acetic, lactic, citric, cinnamic, succinic, fumaric, maleric, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic acid, and the like.

ODV salts used in the preparation of compositions contained in transdermal patches of the present invention may be crystalline or under a polymorphic or amorphous form. Hydrates as well as anhydrous forms of the salts are also encompassed by the present invention.

Several salts of ODV have been prepared, including the fumarate (U.S. Pat. No. 4,535,186) and succinates (U.S. Pat. No. 6,673,838), that have different physicochemical (e.g., solubility, stability and hygroscopy) and biological characteristics than ODV free base. For example, ODV succinate has been shown to exhibit improved solubility, permeability and bioavailability, and its oral administration has been found to result in a lower incidence of nausea, vomiting, diarrhea, abdominal pain, headache, vaso-vagal malaise, and/or trismus than oral administration of venlafaxine, ODV or other salts of ODV.

Selecting a pharmaceutically acceptable salt of ODV for the preparation of a transdermal drug delivery device of the present invention may readily be performed by one of ordinary skill in the art.

II—Transdermal Drug Delivery Devices

A transdermal drug delivery device according to the present invention preferably consists of a patch containing a topical composition of ODV, or a pharmaceutically acceptable salt thereof, that is to be applied to a skin or mucosa surface of a patient.

Patch

Transdermal patches provided by the present invention may be of the reservoir or porous membrane type or of a solid matrix variety (“Transdermal and Topical Drug Delivery Systems”, T. K Ghosh et al. (Eds), 1997, CRC Press, which is incorporated herein by reference in its entirety).

Preferably, the patch components resemble the viscoelastic properties of the skin and conform to the skin during movement to prevent undue shear and delamination. In certain embodiments, the patch has a specific geometric shape such that it corresponds to the conditions of the area of application. Thus, the shape of the patch can be flat or three-dimensional, round, oval, square, and have concave or convex outer shapes. Alternatively, the patch can be segmented by the user into corresponding shapes with or without auxilliary means.

A reservoir type patch design is characterized by a backing film coated with an adhesive, and a reservoir compartment comprising the composition to be delivered (i.e., a topical ODV composition), in the form of a solution, suspension or semi-solid form, that is separated from the skin by a semi-permeable membrane (see, for example, U.S. Pat. No. 4,615,699, which is incorporated herein by reference in its entirety). The adhesive coated backing layer extends around the reservoir's boundaries to provide a concentric seal with the skin and hold the reservoir adjacent to the skin.

In certain preferred embodiments, the reservoir transdermal drug delivery devices of the present invention are constructed according to the Crystal Reservoir Technology developed by Aveva Drug Delivery Systems (Miramar, Fla.). Crystal Reservoir Technology is based on the over-saturation of an adhesive polymer with the drug to be delivered (here ODV, or a pharmaceutically acceptable salt thereof) thus forcing a partial crystallization of the drug. The presence of both molecular solute and solid crystal forms allows for a considerably higher concentration and consistent supply of drug in each patch. As the skin absorbs the molecular solute, crystals re-dissolve to maintain maximum thermodynamic activity at the site of contact. By modifying the concentration of crystals to solute, various patterns of drug release can be achieved.

In other embodiments, the transdermal drug delivery systems of the present invention are matrix patches. A matrix patch generally comprises a matrix containing the topical composition, an adhesive backing film overlay and preferably, a release liner. In some cases, it may be necessary to include an impermeable layer to minimize drug migration to the backing film (see, for example, U.S. Pat. No. 4,336,243, which is incorporated herein by reference in its entirety). The matrix is held against the skin by the adhesive overlay. Examples of suitable matrix materials include, but are not limited to, lipophilic polymers, such as polyvinyl chloride and polydimethylsiloxane, and hydrophilic polymers, such as polyvinylpyrrolidone, polyvinyl alcohol, hydrogels based on gelatin, and polyvinylpyrrolidone/polyethylene oxide mixtures.

In certain preferred embodiments, the transdermal drug delivery devices of the present invention are gel matrix patches such as Aveva Gel Matrix patches (Aveva Drug Delivery Systems, Miramar, Fla.), which do not cause disruption of the stratum corneum during removal, and therefore can be removed and reapplied with minimal skin irritation.

Alternatively, patches of the present invention may be monolithic drug-in-adhesive patches, which are characterized by the inclusion of the ODV topical composition in the skin contacting adhesive layer, a backing film, and preferably, a release liner. In drug-in-adhesive patches, the adhesive layer has two functions: it releases the drug to the skin surface and adheres the matrix to the skin. A drug-in-adhesive delivery system does not require an adhesive overlay and thus the patch size is minimized. Also, drug-in-adhesive type patches are thin and comfortable (see, for example, U.S. Pat. No. 4,751,087, which is incorporated herein by reference in its entirety). Alternatively, such patches may be multi-laminate and further incorporate a semi-permeable membrane between two distinct drug-in-adhesive layers or multiple drug-in-adhesive layers under a single backing film.

Semi-permeable membranes useful with the reservoir or multi-laminate patches of the present invention, include thin non-porous ethylene vinyl acetate films or thin microporous films of polyethylene employed in microlaminate solid state reservoir patches.

Adhesives for use with the drug-in-adhesive type patches are well known in the art and selection is readily accomplished by one of ordinary skill in the art. Three basic types of commonly used adhesives are polyisobutylenes, silicones, and acrylics. Adhesives suitable for use in the present invention preferably function under a wide range of conditions, such as high and low humidity, bathing, sweating, etc. Preferably, the adhesive is physically and chemically compatible with the composition comprising the active agent(s) (i.e., ODV, or a pharmaceutically acceptable salt thereof, and any other additional pharmacologically active agent also present in the composition). Preferably, the adhesive is a composition based on natural or synthetic rubber; a polyacrylate such as polybutylacrylate, polymethyl-acrylate, poly-2-ethylhexyl acrylate, polyvinylacetate; polydimethylsiloxane; or hydrogels (e.g., high molecular weight polyvinylpyrrolidone and oligomeric polyethylene oxide). Preferred adhesives are pressure sensitive adhesives (PSA) that are suitable for long term skin contract. Examples of such PSA include Durotak® adhesives (e.g., Durotak® 2052, National Starch and Chemicals, Bridgewater, N. J.). The adhesive may contain a thickener, such as a silica thickener (e.g., Aerosil, Degussa, Ridgefield Park, N.J.) or a crosslinker, such as aluminum acetaylacetonate.

During storage and prior to use, a laminated patch includes a release liner. Immediately prior to use, this layer is removed from the device so that the drug delivery system may be applied/affixed to the skin. Preferably, the release liner is made from a material impermeable to the drug and vehicle of the composition to be delivered, and is a disposable element that serves only to protect the patch prior to application. Suitable release liners include, but are not limited to, occlusive, opaque, or clear polyester films with a thin coating of pressure sensitive release liner (e.g., silicone fluorsilicone, and perfluorcarbon based polymers).

The backing layer functions as the primary structural element of the transdermal drug delivery system and provides the device with flexibility. The material used for the backing layer should be inert and impermeable to the components of the composition to be delivered. The backing is preferably comprised of a flexible elastomeric material that serves as a protective covering to prevent loss of drug and/or vehicle via transmission through the upper surface of the patch. Furthermore, the material used for the backing layer should permit the device to follow the contours of the skin and be worn comfortably on areas of the skin such as at joints or other points of flexure, that are normally subjected to mechanical strain with little or no likelihood of the device disengaging from the skin due to differences in the flexibility or resiliency of the skin and the device. Backing films may be occlusive or permeable and are preferably derived from synthetic polymers such as polyolefin oils polyester, polyethylene, polyvinylidine chloride, and polyurethane or from natural materials like cotton, wool, and the like. Occlusive backing films, such as synthetic polyesters, result in hydration of the outer layers of the stratum corneum while non-occlusive backing allow the area to breath (i.e., promote water vapor transmission from the skin surface).

Additional layers such as, for example, intermediate fabric layers and/or rate-controlling membranes, may also be present in any of the patch designs described above. Fabric layers may be used to facilitate fabrication of the device, while a rate-controlling membrane may be used to control the rate at which the component(s) of the composition permeate(s) out of the device. A rate controlling membrane, if present, will be included in the system on the skin side of one or more of the drug reservoirs. The materials used to form such as membrane are generally selected to limit the flux of one or more components contained in the topical formulation. Representative materials useful for forming rate-controlling membranes include polyolefins such as polyethylene and polypropylene, polyamides, polyesters, ethylene-vinyl acetate copolymer, ethylene-vinyl methylacetate copolymer, ethylene-vinyl ethylacetate copolymer, ethylene-vinyl polylacetate copolymer, polyisoprene, polyacrylonitrile, ethylene-propylene copolymer, and the like.

As will be obvious to one skilled in the art, components of the composition to be delivered may be contained in separate patches each applied to the patient's body surface. Alternatively, the patch may comprise two or more patch segments each containing different components of the composition to be delivered that are assembled immediately prior to use (for example, one may contain ODV, or a pharmaceutically acceptable salt thereof, and the other may contain one or more additional pharmacologically active agents). Alternatively, a patch of the present invention may comprise two or more reservoirs containing different components to be delivered.

The construction of transdermal delivery systems as described above are known in the art and include conventional coating and laminating techniques (see, for example, “Transdermal Controlled Systemic Medications”, Y. W. Chien (Ed), 1987, Marcel Dekker, Inc.: New York, DE 33 15 272, DE 38 43 239, EP 261 402, and U.S. Pat. No. 3,598,122, each of which is incorporated herein by reference in its entirety). For example, the adhesive matrix systems of the present invention may be prepared by casting a fluid admixture of adhesive and ODV topical composition onto the backing layer, followed by lamination of the release liner. Alternatively, the adhesive mixture may be cast onto the release liner, followed by lamination of the backing layer. Alternatively, the drug reservoir may be prepared in the absence of the topical composition to be delivered, and then loaded by soaking it in the ODV composition.

In addition to offering advantages such as constant rate of administration, improved patient compliance, elimination or reduction of adverse side effects and drug-drug interactions, non-invasive dosing and reversible action (by simply removing the patch), the transdermal drug delivery systems of the present invention also allow for a controlled delivery of specific amounts of ODV, or a pharmaceutically acceptable salt thereof, over a specific period of time. Patches of the present invention may be designed for controlled release of ODV over a couple of hours, 24 hours, 48 hours, 1 week, 1 month, etc. depending on the intended purpose of the patch.

ODV Topical Compositions

The ODV topical compositions contained in the transdermal delivery devices of the present invention are preferably liquid or semi-solid dosage preparations. For example, the ODV compositions may be formulated as solutions, dispersions, suspensions, emulsions, mixtures, lotions, liniments, jellies, ointments, creams, pastes, gels, hydrogels, and foams. The ODV topical compositions may be prepared according to general pharmaceutical practice (see, for example, “Remington's Pharmaceutical Sciences” E. W. Martin, 18^{th} Ed., 1990, Mack Publishing Co.: Easton, Pa., and “Encyclopedia of Pharmaceutical Technology”, J. Swarbrick, and J. C. Boylan (Eds.), Marcel Dekker, Inc: New York, 1988, each of which is incorporated herein by reference in its entirety).

An ODV topical composition generally comprises a therapeutically effective amount of ODV, or a pharmaceutically acceptable salt thereof, and at least one physiologically acceptable carrier, vehicle or excipient. Physiologically acceptable carriers, vehicles, and/or excipients suitable for incorporation into the ODV compositions can be routinely selected for a particular use by one skilled in the art. Such carriers, vehicles, and/or excipients include, but are not limited to, solvents, buffering agents, inert diluents or fillers, suspending agents, dispersing or wetting agents, preservatives, stabilizers, chelating agents, emulsifying agents, anti-foaming agents, gel-forming agents, ointment bases, penetration enhancers, humectants, and emollients.

Examples of solvents are water or purified water, alcohols (e.g., ethanol, benzyl alcohol), vegetable, marine and mineral oils, polyethylene glycols, propylene glycols, glycerol, and liquid polyalkylsiloxanes. Inert diluents or fillers may be sucrose, sorbitol, sugar, mannitol, microcrystalline cellulose, starches, calcium carbonate, sodium chloride, lactose, calcium phosphate, calcium sulfate, or sodium phosphate. Examples of buffering agents include citric acid, acetic acid, lactic acid, hydrogenophosphoric acid, diethylamine, sodium hydroxide and tromethane (i.e., tris(hydroxymethyl)aminomethane hydrochloride). Suitable suspending agents are, for example, naturally occurring gums (e.g., acacia, arabic, xanthan, and tragacanth gum), celluloses (e.g., carboxymethyl-, hydroxyethyl-, hydroxypropyl-, and hydroxypropylmethyl-cellulose), alginates and chitosans. Examples of dispersing or wetting agents are naturally occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate).

Preservatives may be added to a topical composition to prevent microbial contamination that can affect the stability of the formulation and cause infection in the patient. Suitable examples of preservatives include parabens (such as methyl, ethyl, propyl, p-hydroxybenzoate, butyl, isobutyl, and isopropylparaben), potassium sorbate, sorbic acid, benzoic acid, methyl benzoate, phenoxyethanol, bronopol, bronidox, MDM hydantoin, iodopropynyl butylcarbamate, benzalconium chloride, cetrimide, and benzylalcohol. Examples of chelating agents include sodium EDTA and citric acid.

Examples of emulsifying agents are naturally occurring gums, naturally occurring phosphatides (e.g., soybean lecithin, sorbitan mono-oleate derivatives), sorbitan esters, mono glycerides, fatty alcohols (e.g., cetyl alcohol, oleyl alcohol), and fatty acid esters (e.g., triglycerides of fatty acids, sodium cetostearyl sulfate). Anti-foaming agents usually facilitate manufacture of the composition, they dissipate foam by destabilizing the air-liquid interface and allow liquid to drain away from air pockets. Examples of anti-foaming agents include simethicone, dimethicone, ethanol, and ether.

Examples of gel bases or viscosity-increasing agents are liquid paraffin, polyethylene, fatty oils, colloidal silica or aluminum, glycerol, propylene glycol, propylene carbonate, carboxyvinyl polymers, magnesium-aluminum silicates, hydrophilic polymers (such as, for example, starch or cellulose derivatives), water-swellable hydrocolloids, carragenans, hyaluronates, alginates, and acrylates. Ointment bases suitable for use in the compositions contained in the transdermal drug delivery devices of the present invention may be hydrophobic or hydrophilic, and include paraffin, lanolin, liquid polyalkylsiloxanes, cetanol, cetyl palmitate, vegetal oils, sorbitan esters of fatty acids, polyethylene glycols, and condensation products between sorbitan esters of fatty acids, ethylene oxide (e.g., polyoxyethylene sorbitan monooleate), polysorbates, white petrolatum and white wax.

Examples of humectants are ethanol, isopropanol glycerin, propylene glycol, sorbitol, lactic acid, and urea. Suitable emollients include cholesterol and glycerol.

ODV topical compositions may, alternatively or additionally, comprise other types of excipients including, thickening agents, bioadhesive polymers, and permeation enhancing agents.

Thickening agents are generally used to increase viscosity and improve bioadhesive properties of pharmaceutical or cosmetic compositions. Examples of thickening agents include, but are not limited to, celluloses, polyethylene glycol, polyethylene oxide, naturally occurring gums, gelatin, karaya, pectin, alginic acid, povidone, and Carbopol® polymers. Bioadhesive polymers are useful to hydrate the skin and enhance its permeability. Bioadhesive polymers can also function as thickening agents. Examples of bioadhesive polymers include, but are not limited to, pectin, alginic acid, chitosan, polysorbates, poly(ethyleneglycol), oligosaccharides and polysaccharides, cellulose esters and cellulose ethers, and modified cellulose polymers.

Permeation enhancing agents are vehicles containing specific agents that affect the delivery of active components through the skin. Examples of permeation enhancing agents include alcohols (e.g., ethyl alcohol, isopropyl alcohol), dimethyl formamide, dimethyl acetamide, dimethyl sulfoxide, 1-dodecylazocyloheptan-2-one, N-decyl-methylsulfoxide, lactic acid, N,N-diethyl-m-toluamide, N-methyl pyrrolidone, nonane, oleic acid, petrolatum, polyethylene glycol, propylene glycol, salicylic acid, urea, terpenes, and trichloroethanol. Other examples include poly(oxyethylene)-poly(oxypropylene) block copolymers, commercially known as poloxamers; ethoxylated hydrogenated castor oils; polysorbates, such as Tween 20 or Tween 80, cetylpyridinium chloride, betaines and sulfobetaines. Still other examples of suitable permeation enhancers include pentadecalactone, 2-pyrrolidine, 1-dodecal-azacycloheptane-2-one, calcium thioglycolate, hexanol, derivatives of 1,3-dioxanes (i.e., 1,3-dioxacyclohexanes) and 1,3-dioxalanes (i.e., 1,3-dioxacyclopentanes), 1-N-dodecyl-2-pyrrolidone-5-carboxylic acid, 2-pentyl-2-oxo-pyrrolidineacetic acid, 2-dodecyl-2-oxo-1-pyrrolidineacetic acid, and 1-azacycloheptan-2-one-2-dodecylacetic acid among others.

In certain embodiments, the ODV compositions may be formulated to provide a local controlled release of one or more components of the composition. Any pharmaceutically acceptable carrier vehicle or formulation suitable for local administration may be employed. Slow release formulations known in the art include coated-pellets, polymer formulations (such as vesicles or liposomes), microparticles (e.g., microspheres or microcapsules). Methods for the manufacture of coated-pellets, liposomes, microspheres and microcapsules are well known in the art.

A wide variety of biodegradable materials may be used to provide controlled release of one or more components of the ODV compositions. The controlled release material should be biocompatible and be degraded, dissolved or absorbed in situ in a safe and pharmaceutically acceptable manner so that the material is removed from the site of administration by natural tissue processes and in a suitable amount of time (e.g., less than one year, preferably less than six months, and most preferably less than one month). The controlled release carrier should not cause any unwanted local tissue reaction, nor should it induce systemic or local toxicity.

Suitable controlled release biodegradable polymers for use in the formulation of the ODV topical compositions may comprise polylactides, polyglycolides, poly(lactide-co-glycolides), polyanhydrides, polyorthoesters, polycaprolactones, poly-saccharides, polyphosphazenes, proteinaceous polymers and their soluble derivatives (such as gelation biodegradable synthetic polypeptides, alkylated collagen, and alkylated elastin), soluble derivatives of polysaccharides, polypeptides, polyesters, and polyorthoesters.

The pharmacokinetic release profile of these formulations may be first order, zero order, bi- or multi-phasic, to provide the desired therapeutic effect (e.g., pain relief) over the desired period of time. A desired release profile can be achieved by using a mixture of polymers having different release rates and/or different percent loading of ODV, or a pharmaceutically acceptable salt thereof, and of additional pharmacologically active agents.

III—Additional Biologically Active or Therapeutic Agents

The ODV topical compositions contained in the transdermal patches of the present invention can be administered alone to treat major depressive disorder, anxiety disorders, vasomotor symptoms or pain, or they may be combined with one or more pharmacologically active agents. More specifically, transdermal drug delivery devices are provided herein that contain a topical ODV composition as described above further comprising a therapeutically effective amount of at least one of a variety of pharmacologically active agents. As will be obvious to one skilled in the art, the additional pharmacologically active agents to be combined with an ODV composition will be selected based on the intended purpose of the patch.

An ODV topical composition may comprise only one pharmacologically active agent, or, alternatively, it may comprise several active agents. A pharmacologically active agent may exhibit a single desirable property or more than one desirable property.

Pharmacologically active agents suitable for incorporation into the topical compositions of the present invention include, but are not limited to, analgesics, anesthetics, muscle relaxants, neurotransmitter regulating agents, nociceptic agents, pre-menstrual medications, anti-menopausal agents, anti-aging agents, anti-anxiolytic agents, mood disorder agents, anti-depressants, anti-bipolar agents, anti-schizophrenic agents, tranquilizers, soporific agents, anti-migraine agents, skin temperature lowering products, anti-cancer agents, alkaloids, anti-metastatic agents, blood pressure controlling agents, hormones, steroids, anti-inflammatory agents, anti-ischemic agents, anti-arrhythmic agents, vitamins, minerals, anti-angiogenic agents, wound healing agents, cytokines, growth factors, anti-histaminic agents, anti-bacterial agents, anti-viral agents, antibiotics, counteracting appetite suppressants, dermatological agents such as skin renewal agents and emollients, libido altering agents, laxatives, anti-diarrheic agents, antipruritic agents, antipyretic agents, immunostimulating agents, and other agents suitable for the treatment of prophylaxis diseases and conditions associated or accompanied with pain and inflammation. Specific examples of suitable pharmacologically active agents are provided and discussed below.

Pain Relieving Agents

In certain embodiments of the invention, the additional pharmacologically active agent(s) has/have pain-relief activity. Alternatively or additionally, the additional pharmacologically active agents may relieve one or more side effects associated with the pain-relieving agent(s) contained in the patch, or may relieve one or more other symptoms or conditions associated with the pain or otherwise of concern to the subject suffering from or susceptible to pain.

There are two types of pain: nociceptive pain and neuropathic pain. Nociceptive pain has been defined as an appropriate physiological response to a painful stimulus. It is caused by noxious stimulation of peripheral nerve endings (i.e., nociceptors), which then transmit impulses over intact neural pathways to the spinal neurons and then to the brain. Nociceptive pain may occur as a result of inflammation, injury, disease or muscle spasm. Neuropathic pain has been defined as an inappropriate response caused by a primary lesion or dysfunction in the nervous system. It is generally caused by damage to neural structures, mainly to nociceptors, which become extremely sensitive and can generate impulses in the absence of stimulation. Nociceptor damage may be due to, for example, trauma, infection, metabolic disorder or cancer. Neuropathic pain is a major factor in the development of chronic pain, and may be associated with pathological states where there is a reduction in pain threshold (i.e., allodynia), an increased response to noxious stimuli (hyperalgesia), or an increased response duration (persistent pain).

The present invention provides transdermal drug delivery devices containing an ODV topical composition as described above further comprising a therapeutically effective amount of at least one pain-relieving agent. Pain-relievers suitable for incorporation into the ODV topical compositions include, but are not limited to, substances, molecules, agents or drugs which, when applied locally, have a temporary analgesic, anesthetic, numbing, paralyzing, relaxing, and/or calming effect.

Analgesics suitable for use in the present invention include non-steroidal, anti-inflammatory drugs (NSAIDs). NSAIDs have analgesic, antipyretic and anti-inflammatory activity. They act peripherally to provide their analgesic effect by interfering with the synthesis of prostaglandin, through cyclooxygenase (COX) inhibition. There are many different types of NSAIDs, including aspirin and other salicylates. Examples include, but are not limited to, ibuprofen, naproxen, sulindac, diclofenac, piroxicam, ketoprofen, diflunisal, nabumetone, etodolac, oxaprozin, and indomethacin. Aspirin acts as an anti-inflammatory agent when administered in high doses, otherwise it is just a pain killer like acetaminophen. Acetaminophen has similar analgesic and antipyretic effects to the NSAIDs, but does not provide an anti-inflammatory effect. Several of the more potent NSAIDs have been developed into topical products for local applications to painful areas of the body.

Analgesics suitable for use in the present invention also include opioids. As used herein, the term “opioid” refers to any agonists or antagonists of opioid receptors such as the μ-, κ-, and δ-opioid receptors and different subtypes. Some opioids exhibit a high affinity for one of the opioid receptors, while others interact with more than one receptors. Opioids that can be used in the practice of the present invention include all agonists and antagonists with morphine-like activity; naturally occurring endogenous and synthetic opioid peptides; and opiates (i.e., drugs which are derived from opium, such as morphine, codeine and a wide variety of semi-synthetic opioid congeners derived from these compounds and from thebaine, another component of opium).

Examples of suitable opioids include, but are not limited to, alfentanil, allylprodine, alphaprodine, amiphenazole, anileridine, benzeneacetamine, benzoylhydrazone, benzylmorphine, benzitramide, nor-binaltorphimine, bremazocine, buprenorphine, butorphanol, clonitazene, codeine, cyclazocine, desomorphine, dextromoramide, dezocine, diampromide, dihydrocodeine, dihydrocodeine enol acetate, dihydromorphine, dimenoxadol, dimepheptanol, dimethyl-thiambutene, dioxaphetyl butyrate, dipipanone, diprenorphine, eptazocine, ethoheptazine, ethylketocyclazocine, ethylmethylthiambutene, etonitazene, etorphine, fentanyl, hydrocodone, hydromorphone, hydroxypethidine, isomethadone, ketobemidone, levallorphan, levorphanol, lofentanil, loperamide, meperidine, meptazinol, metazocaine, methadone, metopon, morphine, morphiceptin, myrophine, nalbuphine, nalmefene, nalorphine, naltrindole, naloxone, naltrexone, narceine, nicomorphine, norlevorphanol, normethadone, normorphine, norpipanone, opium, oxycodone, oxymorphone, papaveretum, papaverine, pentazocine, phenadoxone, phenazocine, phenoperidine, piminodine, piperidine, pirtramide, proheptazine, promedol, propiram, propoxyphene, remifentanil, spiradoline, sufentanil, tilidine, trifluadom, and active derivatives, prodrugs, analogs, pharmaceutically acceptable salts, or mixtures thereof.

Examples of suitable peptide opioids include, but are not limited to, [Leu⁵]enkephalin, [Met⁵]enkephalin, DynorphinA, Dynorphin B, α-Neoendorphin, β-Neoendorphin, βₕ-Endorphin, Deltorphin II, Morphiceptin, and active derivatives, analogs, pharmaceutically acceptable salts, or mixtures thereof.

Since synergy is known to occur between opioids of different classes (J. U. Adams et al., J. Pharmacol. Exp. Ther., 1993, 266: 1261-1267; L. He and N. M. Lee, J. Pharmacol. Exp. Ther., 1998, 285: 1181-1186; G. C. Rossi et al., Brain Res., 1994, 665: 85-93), in certain embodiments, the topical compositions contained in the inventive patches comprise ODV, or a salt thereof, as described above and a therapeutically effective amount of two or more opioid analgesics.

Opioids are also known to work in combination with other classes of drugs (see, for example, U.S. Pat. Nos. 5,840,731 and 5,869,498; and WO 97/10815). Adjuvant drugs may be used to enhance the analgesic efficacy of opioids, treat concurrent symptoms that exacerbate the pain, or provide independent analgesia for specific types of pain. Agents that may be used as adjuvant drugs include, but are not limited to, local anesthetics, antidepressants, anticonvulsants, and corticosteroids.

Anesthetics, such as xylocalne, lidocaine or benzocaine (or other drugs such as those described below) may be added to the inventive ODV topical compositions to provide an immediate but short-term pain relief until ODV and/or another analgesic becomes fully effective.

Anesthetics that are suitable for use in the practice of the present invention include sodium-channel blockers. Sodium-channel blockers prevent the generation and conduction of nerve impulses by decreasing or preventing the large transient increase in the permeability of excitable membranes to sodium ions, Na⁺. Examples of sodium-channel blockers include, but are not limited to, ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, etidocaine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocalne, procaine, propanocaine, proparacaine, propipocaine, propoxycaine, pseudococaine, pyrrocaine, ropivacaine, salicyl alcohol, tetracaine, tolycaine, trimecaine, zolamine, and active derivatives, prodrugs, analogs, pharmaceutically acceptable salts, or mixtures thereof.

In order to improve the effectiveness and tolerance of the topical composition, local anesthetics with different pharmacodynamics and pharmacokinetics may be combined in the composition. Accordingly, in certain embodiments, the composition comprises ODV, or a salt thereof, as described above, and a therapeutically effective amount of two or more anesthetic agents. For example, a preferred combination of anesthetic agents is an eutectic mixture of lidocaine and prilocalne. Another preferred combination is a mixture of lidocaine and tetracaine.

In other embodiments, the ODV topical composition further comprises a therapeutically effective amount of an agent that can prolong the local anesthetic effect and/or enhance the effectiveness of the local anesthetic agent(s) contained in the composition.

It has been reported (see, for example, U.S. Pat. Nos. 5,922,340 and 6,046,187) that the co-administration of a glucocorticosteroid may prolong or otherwise enhance the effect of local anesthetics. Glucocorticosteroids that may be used in the ODV compositions include dexamethazone, cortisone, hydrocortisone, prednisone, prednisolone, beclomethasone, betamethasone, flunisolide, fluocinolone, acetonide, fluocinonide, triamcinolone, and the like.

Locally acting vasoconstrictive agents are also known to provide effective enhancement of local anesthesia, especially when administered through controlled release. Vasoconstrictor agents include, but are not limited to, catechol amines (e.g. epinephrine, norepinephrine and dopamine); metaraminol, phenylephrine, sumatriptan and analogs, α-1 and α-2 adrenergic agonists, such as, for example, clonidine, guanfacine, guanabenz, and dopa (i.e., dihydroxyphenylalanine), methyldopa, ephedrine, amphetamine, methamphetamine, methylphenidate, ethylnorepinephrine ritalin, pemoline, and other sympathomimetic agents.

Other adjuvant drugs that can be used in the present invention include N-methyl-D-aspartate (“NMDA”) receptor antagonists (such as ketamine), which are known to have local anesthetic properties. In addition to ketamine, NMDA-receptor antagonists include dextro-methorphan, dextrorphan, pyroloquinoline quinone, cis-4-(phosphonomethyl)-2-piperidine carboxylic acid, MK801, and memantine.

Anti-Inflammatory Agents

Inflammation is a natural consequence of injury of adult tissues and the body's initial attempt at healing itself. While the inflammatory response is essential to healing, severe, prolonged inflammation can perpetuate pain. The present invention provides transdermal drug delivery devices containing ODV topical compositions further comprising a therapeutically effective amount of at least one anti-inflammatory agent. Anti-inflammatory agents for use in the present invention are substances, molecules, agents or drugs, which, when applied topically, have an anti-inflammatory activity (i.e., they can prevent or reduce the duration and/or severity of inflammation; prevent or reduce injury to cells or damage to tissue caused by inflammation; and/or provide relief from at least one of the manifestation of inflammation such as erythema, swelling, tissue ischemia, itching, fever, and the like).

Anti-inflammatory agents suitable for use in the present invention may be selected from a wide variety of steroidal and non-steroidal anti-inflammatory agents.

Examples of NSAIDs can be found above. Examples of steroidal anti-inflammatory agents include, but are not limited to, aclomethasone dipropionate, flunisolide, fluticasone, budesonide, triamcinolone, triamcinoline acetonide, beclomethasone diproprionate, betamethasone valerate, betamethasone diproprionate, hydrocortisone, cortisone, dexamethason, mometasone furoate, prednisone, methylprednisolone aceponate, and prednisolone. Steroids are synthetic forms of naturally occurring hormones produced by the adrenal glands. They can provide rapid and powerful reduction of pain and inflammation by stopping the production of prostaglandins. Local administration of steroids avoids the side effects which are generally associated with their systemic administration including blood sugar elevations, hypertension, osteoporosis, and weight gain.

Alternatively or additionally, anti-inflammatory agents may be selected from the wide variety of substances, molecules, and drugs exhibiting antioxidant activity. Antioxidants are agents that can prevent or reduce oxidative damage caused to tissue by inflammatory processes that involve the production of reactive oxygen species (ROS). Antioxidants suitable for incorporation in the ODV topical compositions of the present invention are substances, molecules or drugs that can prevent, inhibit or suppress biological damage associated with reactive oxygen species. These include agents that can scavenge ROS; agents that can limit the production of ROS by activated neutrophils or macrophages, for example, by inhibiting the respiratory burst; agents that can reduce the number of neutrophils or macrophages attracted to the site of inflammation; and agents that effect their antioxidant activity by any combinations of these mechanisms of action.

Antioxidants may be selected from the group consisting of vitamin A (retinal), vitamin B (3,4-didehydroretinol), vitamin C (D-ascorbic acid, L-ascorbic acid), α-carotene, β-carotene, γ-carotene, δ-carotene, vitamin E (α-tocopherol), β-tocopherol, γ-tocopherol, δ-tocopherol, tocoquinone, tocotrienol, butylated hydroxy anisole, cysteine, and active derivatives, analogs, precursors, prodrugs, pharmaceutically acceptable salts or mixtures thereof.

An anti-inflammatory ODV topical composition contained in a transdermal drug delivery device of the invention may further comprise a topical antipruritic agent such as menthol, and/or a decongestant such as eucalyptus oil.

Anti-Cancer Agents

As already mentioned above, cancer is often associated with pain. Accordingly, the present invention provides transdermal patches containing ODV topical compositions further comprising a therapeutically effective amount of at least one chemotherapeutic anti-cancer agent. These inventive transdermal patches may, for example, be applied to a surgical site from which a tumor has been ablated to alleviate pain and prevent regrowth from any residual tumor cells after closure of the surgical wound.

Chemotherapeutic anti-cancer agents suitable for incorporation in the ODV topical compositions are substances, molecules, agents or drugs which, when applied topically, can prevent or reduce cancer cell proliferation, destroy cancer cells, and/or prevent or reduce metastasis.

Examples of chemotherapeutic anti-cancer agents include, but are not limited to, alitretinoin, altretamine, bexarotene, capecitabine, carmustine with Polifeprosan 20 Implant (Gliadel Wafer), cisplatin, cytarabine liposomal (DepoCyt), cyclophosphamide, daunorubicin liposomal, docetaxel, doxorubicin liposomal, epirubin, etoposide phosphate, 5-fluorouracil, gemcitabine, gemtuzumab-ozogamicin, imatinib mesylate (Gleevec), irinotecan, oxaliplatin, levamisole, navelbine, mitoguazone, mitomycin, mitoxantrone, paclitaxel, temozolamide, topotecan, triapine, trimetrexate, somatuline, valrubicin, and vinblastine.

Other Pharmacologically Active Agents

In other embodiments of the invention, the additional pharmacologically active agent is selected for its ability to directly or indirectly prevent, alleviate or reduce vasomotor symptoms.

Vasomotor symptoms (VMS), which include hot flashes and night sweats, are the most common symptoms associated with menopause, occurring in 60% to 80% of all women following natural or chemically- or surgically-induced menopause (H. L. Judd et al., Obstet. Gynecol., 1981, 58: 267-275). A hot flash is characterized by a heat-dissipation response that consists of the sudden onset of sweating of the face, neck and chest, as well as peripheral withdrawal vasodilation (R. R. Freedman, Am. J. Human Biol., 2001, 13: 453-464). Hot flashes can last up to 30 minutes and vary in their frequency from several times a week to multiple occurrences per day. Often dizziness, palpitations and diaphoresis accompany such episodes, which can lead to sleep disruption and interfere with the quality of life. Vasomotor symptoms are often even more severe in women treated for breast cancer, in particular in those patients who are given the anti-estrogen drug tamoxifen. Men also experience hot flashes following steroid hormone (androgen) withdrawal, in cases of age-associated androgen decline as well as in extreme cases of hormone deprivation associated with treatment for prostate cancer (H. H. Berendsen et al., Eur. J. Pharmacol., 2001, 419: 47-54). As many as one-third of these prostate cancer patients experience persistent and frequent symptoms severe enough to cause significant discomfort and inconvenience.

In those embodiments where the transdermal drug delivery devices of the present invention are to be used in the management of vasomotor symptoms or vasomotor instability, the ODV composition contained in the device may further comprise a therapeutically effective amount of at least one pharmacologically active agent selected for its ability to prevent, reduce or alleviate one or more vasomotor symptoms. Alternatively or additionally, a pharmacologically active agent may be selected for its ability to relieve one or more other symptoms or conditions associated with VMS or otherwise of concern to the subject suffering from VMS.

The most commonly used treatments for hot flashes are hormone-replacement therapy (HRT; estrogen and progesterone) and estrogen-replacement therapy (ERT). Accordingly, in certain embodiments, the ODV topical compositions of the present invention further comprise a therapeutically effective amount of at least one hormone known to be useful in the management of vasomotor symptoms. Suitable hormones include estrogens, progestins, and androgens.

The term “estrogen”, as used herein, refers to any substance, natural or synthetic, that exerts a biological or pharmacological action primarily by binding to estrogen receptors. Examples of suitable estrogens include, but are not limited to, 17-β-estradiol, 17-α-estradiol, estriol, estrone, and phytoestrogens. These substances may be derived or modified to form, for example, conjugated estrogens, esterified estrogens, ethinyl estradiol, etc. Also suitable are selective estrogen receptor modulators such as raloxifene and the like. Estrogenic hormones incorporated into the ODV topical compositions may be present as salts (e.g., sodium estrogen sulfate), isomers, or prodrugs. Examples of phytoestrogens (i.e., plant-derived estrogens) include isoflavones such as genistein, diaszein and equol.

The term “progestin”, as used herein, refers to any substance, natural or synthetic, that exerts a biological or pharmacological action primarily by binding to progestin receptors. Examples of suitable progestins for use in the patches of the present invention include, but are not limited to, progesterone, medroxy-progesterone acetate, norethindrone, and norethindrone acetate, esters, derivatives, prodrugs, and isomers thereof.

The term “androgen”, as used herein, refers to a steroid, natural or synthetic, which exerts its biological or pharmacological action primarily by binding to androgen receptors. Examples of suitable androgens for incorporation into the ODV topical compositions include, but are not limited to, testosterone, methyltestosterone, androstenedione, adrenosterone, dehydroepiandrosterone, oxymetholone, fluoxymesterone, methandrostenolone, testolactone, pregnenolone, 17α-methylnortestosterone, norethandrolone, dihydrotestosterone, danazol, androsterone, nandrolone, stanozolol, ethylestrenol, oxandrolone, bolasterone, mesterolone, testosterone propionate, testosterone cypionate, testosterone phenylacetate, and testosterone enanthate, testosterone acetate, testosterone buciclate, testosterone heptanoate, testosterone decanoate, testosterone caprate, testosterone isocaprate, as well as esters, derivatives, prodrugs, and isomers thereof.

Although hormonal treatments are very effective at alleviating VMS, they are not appropriate for all patients. In particular, hormone therapy is usually not recommended for patients with or at risk for hormonally sensitive cancers (e.g., breast or prostate cancer). Furthermore, patients with history of clotting or severe migraines are averse to undergoing hormonal therapy because other estrogen-mediated side effects (e.g., uterine cancer, vaginal bleeding, and vein thrombosis) may emerge. Accordingly, in certain embodiments, the transdermal drug delivery devices of the present invention contain an ODV topical composition further comprising one or more non-hormonal pharmacologically active agents. Examples of non-hormonal agents suitable for incorporation into the inventive ODV topical compositions include, but are not limited to, steroids, α-adrenergic agonists, and β-blockers. Specific examples include bellargal (i.e., a combination of phenobarbital, ergotamine, and belladonna; T. B. Lebherz, Obstet. Gynecol., 1969, 33: 795-799), clonidine (R. M. Goldberg et al., J. Clin. Onc., 1994, 12: 155-158; C. L. Loprinzin et al., J. Urol., 1994, 151: 634-636), mirtazapine (M. D. Waldinger et al., Maturitas, 2000, 36: 165-168), trazadone (F. Pansini et al., Clin. Exp. Obstet. Gynecol., 1995, 22: 341-344), gabapentin (T. J. Guttuso, Neurology, 2000, 54: 2161-2163), veralipride (A. David, Am J Obstet. Gynecol., 1988, 158:1107-1115: P. Vercellini et al., Gynecol. Obstet. Invest., 1992, 34: 102-104), methyldopa (M. G. Hammond, J. Clin. Endocrinol. Metab., 1984, 58: 1158-1160; O. Andersen, Acta Obstet. Gynecol. Scand., 1986, 65: 405-409; B. I. Nesheim, Eur. J. Clin. Pharmacol., 1981, 20: 413-416.), bromocryptine (B. Scoccia et al., J. Clin. Endocrinol. Metab, 1988, 66: 868-871), and domperidone (L. Zichella et al., Maturitas, 1986, 8: 229-237).

A more complete list of pharmacologically active compounds and substances suitable for incorporation into ODV topical compositions contained in transdermal patches of the present invention can be found in the “Physicians' Desk Reference”, 55^{th} Ed., 2001 Medical Economics Co., Inc.: Montvale, N. J., which is incorporated herein by reference in its entirety. For most or all of these agents, recommended effective dosages and regimes are known in the art.

IV—Uses of ODV Topical Compositions

According to the present invention, the transdermal patches provided herein are useful for treating a variety of diseases, disorders or conditions. In particular, the inventive transdermal patches can be used for the treatment of depression and anxiety disorders and for the prevention, treatment or management of vasomotor symptoms and pain.

In certain embodiments, the transdermal drug delivery systems of the present invention are used for treating female patients experiencing vasomotor instability associated with either natural menopause resulting from age-related declining ovarian function or premature or artificially-induced menopause secondary to an ovariectomy, breast cancer treatment, x-ray radiation, etc. In other embodiments, the transdermal patches are used for treating male patients experiencing vasomotor symptoms associated with either age-related androgen decline or hormone deprivation resulting from treatment for prostate cancer. In still other embodiments, the inventive transdermal patches are used to treat any male or female individual experiencing VMS not associated with menopause or androgen decline.

Alternatively or additionally, the drug delivery devices of the present invention may be used to treat any of a variety of different types of pain experienced by mammals, including humans. For example, the inventive patches may be used to treat acute pain (short duration) or chronic pain (regularly reoccurring or persistent), whether centralized or peripheral.

Examples of pain that can be acute or chronic and that can be treated in accordance with the methods of the present invention include inflammatory pain, musculoskeletal pain, bony pain, lumbosacral pain, neck or upper back pain, visceral pain, somatic pain, neuropathic pain, cancer pain, pain caused by injury or surgery such as burn pain, or headaches such as migraines or tension headaches, or combinations of these pains. One skilled in the art will recognize that these pains may overlap one another. For example, a pain caused by inflammation may also be visceral or musculoskeletal in nature.

In certain embodiments, the transdermal patches of the present invention are used to treat or prevent pain related to or induced by any one of the following diseases, trauma or conditions: general neuropathic conditions, such as peripheral neuropathy, phantom pain, reflex-sympathetic, dystrophy, causalgia, syringomyelia, and painful scar; specific neuralgias at any location of the body, back pain, diabetic neuropathy, alcoholic neuropathy, metabolic neuropathy; inflammatory neuropathy; chemotherapy-induced neuropathy, herpetic neuralgias, traumatic ondotalgia; endodontic odontalgia; thoracic-outlet syndrome; cervical, thoracic or lumbar radiculopathies with nerve compression; cancer with nerve invasion; traumatic-avulsion injuries; mastectomy, thoracotomy pain; spinal-cord injury; stroke; abdominal-cutaneous nerve entrapments; tumors of neural tissues; arachnoiditis; stump pain; fibromyalgia; regional sprains or strains; myofascial pain; psoriatic arthropathy; polyarteritis nodosa; osteomyelitis; burns involving nerve damage; AIDS-related pain syndromes; connective tissue disorders, such as systemic lupus erythematosis, systemic sclerosis, polymyositis, and dermatomyositis; and inflammatory conditions, such as acute inflammation (e.g., trauma, surgery and infection) or chronic inflammation (e.g., arthritis and gout).

In other embodiments, the transdermal drug delivery devices of the present invention are used for the treatment of diseases and conditions of the central nervous system, in particular those diseases and conditions where serotonin and/or norepinephrine is/are implicated.

For example, the transdermal patches provided by the present invention may be used for treating depression disorders including, but not limited to, depression in cancer patients, depression in Parkinson' patients, postmyocardial infarction depression, subsyndromal symptomatic depression, depression in infertile women, pediatric depression, major depression, single episode depression, recurrent depression, child abuse induced depression, and post-partum depression. Alternatively or additionally, the inventive patches may be used in the treatment of generalized anxiety disorder, phobias, agoraphobia, social phobia and simple phobias, post-traumatic stress syndrome, acute stress disorder, avoidant personality disorder, eating disorders, anorexia nervosa and bulimia nervosa, obesity, obsessive compulsive disorder, panic disorder, premenstrual syndrome, attention deficit hyperactivity disorder. The inventive patches may also be useful in the treatment of borderline personality disorder, schizophrenia and other psychotic disorders, mood disorder associated with psychotic disorders such as acute mania and depression associated with bipolar disorder, and mood disorders associated with schizophrenia.

As will be obvious to one skilled in the art, the compositions of the present invention may be administered alone, or, alternatively, they may be administered serially or in combination with conventional therapeutics or therapeutic regimens used in the treatment of vasomotor symptoms or pain.

V—Dosage and Administration

A patch of the present invention may be applied to a skin or mucosal surface adjacent to a body area to be treated (e.g., area experiencing pain) for local delivery of the ODV composition and minimal absorption of the active ingredient(s) into the subject's bloodstream (e.g., to avoid or reduce systemic effect). Alternatively, local application of a patch of the present invention to a skin or mucosal surface of a patient may result in absorption of at least one active ingredient of the ODV composition into the patient's bloodstream for systemic drug distribution.

Dosage

Dosage of the topical ODV compositions contained in the transdermal drug delivery devices of the present invention will be such that the amount of ODV (or pharmaceutically acceptable salt thereof) delivered is effective for its intended purpose (e.g., prevents, reduces or alleviates pain, or relieves vasomotor symptoms). As will be obvious to one skilled in the art, the dosage will be dependent upon the nature of the condition to be treated (major depression disorder, anxiety disorder, vasomotor symptoms or pain), the severity of the condition, the age, weight, and general health condition of the patient as well as upon the potency, bioavailability, and in vivo half-life of the active ingredient(s) of the topical composition used. These factors are readily determinable by the attending physician in the course of therapy. Alternatively or additionally, the dosage to be administered can be determined from studies using animal models for the particular type of condition being treated, and/or from animal or human data obtained from agents which are known to exhibit similar pharmacological activities. The total dose required for each treatment may be administered by multiple doses or a single dose. Adjusting the dose to achieve maximal efficacy based on these or other methods are well known in the art and are within the capabilities of trained physicians. As studies are conducted, further information will emerge regarding the appropriate dosage levels and duration of treatment of vasomotor symptoms, different types of pain, and other conditions that can benefit from the inventive topical compositions.

In certain embodiments, the patches of the present invention are formulated such that the amount of ODV, or pharmaceutically acceptable salt thereof, to be delivered is between about 5 mg and about 500 mg of ODV, or a pharmaceutically acceptable salt thereof, wherein the amount is calculated based on the amount of ODV free base. For example, the patch may contain between about 25 mg and about 250 mg of ODV or salt thereof, or about 50 mg and about 200 mg of ODV or salt thereof, or about 100 mg ODV or salt thereof, as calculated based on the amount of ODV free base.

The amount of additional pharmacologically active agents (e.g., analgesic or anti-inflammatory agents) present in a transdermal patch of the present invention may vary depending upon the dosage recommended or permitted for the particular agent, as well as the type of condition treated and the presence and nature of other active ingredients in the composition to be delivered. In general, the amount of a pharmacologically active agent present is the ordinary dosage required to obtain the desired result through topical administration. Such dosages are either known to or readily determined by the skilled practitioner in the pharmaceutical and/or medical arts.

Administration

Generally, transdermal patches provided by the present invention are applied to a skin or mucosa surface area, preferably adjacent to the site of interest (e.g., area of the body experiencing pain, or in the lower neck or head to increase absorption of ODV or its salts near the brain for the treatment of depression or anxiety disorders). In certain embodiments, the patch is worn without interruption for a specific period of time (e.g., until most of the ODV composition contained in the patch has been delivered). In other embodiments, the patch is worn only when needed, for example, in the prevention, treatment or management of vasomotor symptoms or pain.

### OTHER EMBODIMENTS

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A transdermal patch for the administration of a topical composition, the topical composition comprising a therapeutically effective amount of ODV, or a pharmaceutically acceptable salt thereof, and at least one physiologically acceptable carrier or excipient.

2. The transdermal patch of claim 1, wherein the topical composition further comprises at least one absorption enhancer.

3. The transdermal patch of claim 2, wherein the absorption enhancer is selected from the group consisting of pentadecalactone, 1,3-dioxalanes, 1,3-dioxanes, and any combination thereof.

4. The transdermal patch of claim 1, wherein the at least one physiologically acceptable carrier or excipient is selected from the group consisting of tromethane ethanol, polyethylene glycol, glycerin, propylene glycol, acrylates, Carbopol, purified water, benzyl alcohol, cetyl alcohol, citric acid, monoglycerides, diglycerides, triglycerides, oleyl alcohol, sodium cetostearylsulphate, sodium hydroxide, stearyl alcohol, white petrolatum, mineral oil, propylene carbonate, white wax, paraffin, and any combination thereof.

5. The transdermal patch of claim 1, 2, or 4, wherein the topical composition further comprises a therapeutically effective amount of at least one pharmacologically active agent.

6. The transdermal patch of claim 5, wherein the at least one pharmacologically active agent is selected from the group consisting of analgesics, anesthetics, muscle relaxants, neurotransmitter regulating agents, nociceptic agents, pre-menstrual medications, anti-menopausal agents, anti-aging agents, anti-anxiolytic agents, mood disorder agents, anti-depressants, anti-bipolar agents, anti-schizophrenic agents, tranquilizers, soporific agents, anti-migraine agents, skin temperature lowering products, anti-cancer agents, alkaloids, anti-metastatic agents, blood pressure controlling agents, hormones, steroids, anti-inflammatory agents, anti-ischemic agents, anti-arrhythmic agents, vitamins, minerals, anti-angiogenic agents, wound healing agents, cytokines, growth factors, anti-histaminic agents, anti-bacterial agents, anti-viral agents, antibiotics, counteracting appetite suppressants, dermatological agents such as skin renewal agents, sun screen and emollients, libido altering agents, laxatives, anti-diarrheic agents, antipruritic agents, antipyretic agents, immunostimulating agents, agents suitable for the treatment of prophylaxis diseases and conditions associated or accompanied with pain and inflammation, and any combination thereof.

7. The transdermal patch of claim 1, wherein the patch is a reservoir patch.

8. The transdermal patch of claim 1, wherein the patch is a matrix patch.

9. The transdermal patch of claim 1, wherein the patch is a drug-in-adhesive patch.

10. The transdermal patch of claim 7, 8 or 9 further comprising a release liner.

11. The transdermal patch of claim 1, 2 or 4, wherein the therapeutically effective amount of ODV, or a pharmaceutically acceptable salt thereof, is between about 5 mg and about 500 mg, between about 25 mg and about 250 mg, or between about 50 mg and about 200 mg, wherein the amount is calculated based on the amount of ODV free base.

12. The transdermal patch of claim 1, 2 or 4, wherein the therapeutically effective amount of ODV, or a pharmaceutically acceptable salt thereof, is about 100 mg, wherein the amount is calculated based on the amount of ODV free base.

13. A method of treating a depression disorder in a subject, the method comprising applying a transdermal patch of claim 1, 2 or 4 to the skin surface of the subject for a period of time effective to treat the depression disorder.

14. The method of claim 13, wherein the depression disorder is selected from the group consisting of major depressive, depression in cancer patients, depression in Parkinson's patients, postmyocardial infarction depression, subsyndromal symptomatic depression, depression in infertile women, pediatric women, single episode depression, recurrent depression, child abuse induced depression and, and post-partum depression.

15. The method of claim 13, wherein the period of time effective to treat the depression disorder is about 1 week to about 1 month.

16. A method of treating an anxiety disorder in a subject, the method comprising applying a transdermal patch of claim 1, 2 or 4 to the skin surface of the subject for a period of time effective to treat the anxiety disorder.

17. The method of claim 16, wherein the anxiety disorder is selected from the group consisting of generalized anxiety disorder, phobias, agoraphobia, social phobia, simple phobias, post-traumatic stress, syndrome, acute stress disorder, avoidant personality disorder, eating disorders, anorexia nervosa, bulimia nervosa, obesity, obsessive compulsive disorder, panic disorder, premenstrual syndrome, and attention deficit disorder.

18. The method of claim 16, wherein the period of time effective to treat the anxiety disorder is about 1 week to about 1 month.

19. A method for treating vasomotor symptoms in a subject, the method comprising applying a transdermal patch of claim 1, 2 or 4 to the skin surface of the subject for a period of time effective to treat vasomotor symptoms.

20. The method of claim 19, wherein the subject suffering from vasomotor symptoms experiences hot flashes.

21. The method of claim 19, wherein the period of time effective to treat vasomotor symptoms is about 30 minutes to about 3 hours.

22. The method of claim 19, wherein the subject is a female patient, and the vasomotor symptoms are associated with natural menopause, chemically-induced menopause or surgically-induced menopause.

23. The method of claim 19, wherein the subject is a female patient who is receiving or has received breast cancer treatment.

24. The method of claim 23, wherein the breast cancer treatment comprises administration of tamoxifen.

25. The method of claim 19, wherein the subject is a male patient who is naturally, chemically or surgically andropausal.

26. The method of claim 25, wherein the male patient is or has been treated for prostate cancer.

27. A method of treating pain in a subject, the method comprising applying a transdermal patch of claim 1, 2 or 4 to the skin surface of the subject for a period of time effective to treat the pain.

28. The method of claim 27, wherein the period of time effective to treat the pain is about 1 hour to about 1 month.

29. The method of claim 28, wherein the transdermal patch is applied to the skin surface adjacent to the subject's body site experiencing pain.

30. The method of claim 29, wherein the pain experienced by the subject is nociceptive pain.

31. The method of claim 29, wherein the pain experienced by the subject is neuropathic pain.
